(19) United States
(12) Patent Application Publication
(10) Pub. No.: US 2016/0296695 A1
(43) Pub. Date: Oct. 13, 2016

(54) COMBINED MEDICAMENT FILL AND CANNULA INSERTION DEVICE

(71) Applicant: Cequr SA, Horw (CH)

(72) Inventors: Michael Hassman, Foxboro, MA (US); Douglas Lawrence, Framingham, MA (US); David Freed, Hopkinton, MA (US); Patrick McDermott, Oxford, MA (US); Stephen Putnam, Littleton, MA (US); Mads Dall, Hellerup (DK); Peter Gravesen, Nordborg (DK); Christian B. Eriksen, Sonderborg (DK); Heiko Arndt, Flensborg (DE)

(21) Appl. No.: 15/094,417

(22) Filed: Apr. 8, 2016

Related U.S. Application Data
(60) Provisional application No. 62/144,620, filed on Apr. 8, 2015.

(51) Int. Cl. A61M 5/158; A61J 1/20; A61M 5/142

(52) U.S. Cl. CPC A61M 5/158; A61M 5/14248; A61J 1/20; A61M 2005/14252; A61M 2005/1585

(57) ABSTRACT

A device for filling a medicament delivery device (e.g., a patch pump) with medicament or fluid and inserting a cannula subcutaneously enables improved results and user safety, while simplifying the set up process for a user. The device can include a housing with a first chamber portion having a first deployment mechanism and a second chamber portion having a second deployment mechanism. The device can also include a trigger mounted to the housing to activate at least one of the first deployment mechanism and the second deployment mechanism and an interface adapted to mate releasably with the medicament delivery device. The filling of the device with medicament and the insertion of the cannula may occur sequentially or simultaneously, and both actions can be initiated by a single user step or a limited number of user steps, as compared to conventional approaches.

### CROSS-REFERENCE TO RELATED APPLICATION

This application claims priority to and the benefit of, and incorporates herein by reference in its entirety, U.S. Provisional Patent Application No. 62/144,620, which was filed on Apr. 8, 2015.

### TECHNICAL FIELD OF THE INVENTION

The invention relates generally to wearable infusion pump devices (e.g., patch pumps) that are filled with medicament and use a cannula for subcutaneous delivery of medicament to a patient. More particularly, the invention relates to an improved technique for filling the pump device and inserting a cannula into the patient, that features a simpler process for a user, improved safety, as well as other advantages.

### BACKGROUND

Infusion devices can be used to deliver medicament (e.g., insulin) or other fluid to a user. Such devices often have an exposed septum that can be pierced by a hollow needle of a syringe, allowing for the filling of the device with medicament. This can be a challenging process involving the handling of sharp objects (e.g., a syringe needle) in a controlled manner requiring a level of dexterity not exhibited by many users. Often, a user must procure a vial of medicament, draw the medicament into the syringe, precisely pierce the septum to empty the medicament from the syringe into an internal reservoir of the device, and remove the syringe from the device thereafter.

Separately, infusion devices generally require a cannula to be placed in the subcutaneous tissue of the patient to establish a fluidic connection between the internal reservoir and the patient. This generally requires the patient to either pierce their own skin with a needle attached to the cannula and to withdraw the needle thereafter, or to activate a device that performs some or all of this process automatically. Conventional devices do not allow for filling the infusion device with medicament and inserting the cannula with a single activation of a single trigger by a user. Rather users must fill the device with medicament and then, in an entirely separate process, insert the cannula into the patient. In some instances the filling and inserting steps require access to opposite sides of the infusion device, which requires that the device be reoriented between performance of these steps. Not only does such a prolonged set-up process cause inconvenience for the user, but it also increases the likelihood that errors will occur in the performance of one or both processes.

### SUMMARY OF THE INVENTION

It is therefore desirable to provide a device that makes performance of the medicament filling and cannula inserting processes safer and easier for a user. As such, one objective of the invention is to provide an apparatus that requires only a single activation (or a limited number of coordinated activations) by the user to fill a medicament delivery device with medicament or other fluid and subcutaneously insert a cannula into the patient. Such an apparatus improves the user experience by reducing and simplifying the steps required to administer medicament. Additionally, overall safety is improved, as, for example, the user may never need to handle an exposed sharp and the apparatus can ensure safe completion of the filling and cannula inserting steps.

In one aspect, the invention relates to an apparatus for filling a medicament delivery device with medicament and inserting a cannula subcutaneously. The apparatus includes a housing forming a first chamber portion containing a first deployment mechanism and a second chamber portion containing a second deployment mechanism. The apparatus also includes a trigger mounted to the housing to activate at least one of the first deployment mechanism and the second deployment mechanism. The apparatus further includes an interface adapted to mate releasably with the medicament delivery device.

In one embodiment of the above aspect, the medicament delivery device is an insulin patch pump attachable to skin of a user. The first chamber may be adapted to receive a medicament container. In some embodiments, the first deployment mechanism includes a first driving member, which may include a first piston portion. In certain embodiments, the second deployment mechanism includes a second driving member, which may include a drive portion. The interface may define a first outlet corresponding with the first chamber portion and a second outlet corresponding with the second chamber portion.

In some embodiments of the above aspect, the trigger is configured to activate both the first deployment mechanism and the second deployment mechanism simultaneously, while in other embodiments the trigger is configured to activate the first deployment mechanism and the second deployment mechanism sequentially. The trigger may be a variety of elements, including a manually actuated button.

In another aspect, the invention relates to a medicament filling and cannula insertion system. The system includes a medicament delivery device and a medicament filling and cannula delivery apparatus. The medicament filling and cannula insertion apparatus includes a first deployment mechanism for deploying a medicament container to fill the medicament delivery device with medicament and a second deployment mechanism for deploying a cannula subcutaneously to a user through the medicament delivery device.

In some embodiments of the above aspect, the medicament filling and cannula insertion apparatus includes a housing forming a first chamber portion housing the first deployment mechanism and a second chamber portion housing the second deployment mechanism, along with an interface adapted to mate releasably with the medicament delivery device. The interface may define a first outlet corresponding with the first chamber portion and a second outlet corresponding with the second chamber portion. The first and second outlets can be arranged to correspond respectively with first and second inlets of the medicament delivery device when the apparatus is releasably attached thereto. The first inlet may be a medicament fill port, which may include a spring and a fill needle. The spring may bias an interface adapted to prevent inadvertent contact with the needle. In certain embodiments, the second inlet is a cannula delivery port, which may include a cannula, a needle, and a spring. The spring may be biased to withdraw the needle after subcutaneous insertion of the cannula.

In another aspect, the invention relates to a method for filling a medicament delivery device and subcutaneously inserting a cannula into a patient. The method includes the step of manually activating a trigger, which results in automatically filling the medicament delivery device with medicament and automatically inserting subcutaneously a cannula into the patient through the medicament delivery device.

In some embodiments of the above aspect, the step of manually activating the trigger includes the patient pushing a button. In certain embodiments, the step of filling the medicament delivery device includes guiding a fill needle of the medicament delivery device into a medicament container. The step of filling the medicament delivery device may include forcing medicament from the medicament container into the medicament delivery device through the fill needle. The step of inserting subcutaneously a cannula may include advancing the cannula and a cannula needle through the skin of the patient. The step of inserting subcutaneously a cannula may further include retracting the cannula needle from the cannula.

In certain embodiments of the above aspect, the steps of filling the medicament delivery device and inserting subcutaneously the cannula occur sequentially. In some cases, the step of filling the medicament delivery device may be completed prior to inserting subcutaneously the cannula. In other embodiments, the steps of filling the medicament delivery device and inserting subcutaneously the cannula may occur simultaneously.

### BRIEF DESCRIPTION OF THE FIGURES

In the drawings, like reference characters generally refer to the same parts throughout the different views. Also, the drawings are not necessarily to scale, emphasis instead generally being placed upon illustrating the principles of the invention. In the following description, various embodiments of the present invention are described with reference to the following drawings, in which:

FIG. 1 is a schematic diagram of an example fill/insert device before being loaded with a medicament vial, according to a first embodiment;

FIG. 2 is a schematic diagram of the example fill/insert device after being loaded with a medicament vial, according to the first embodiment;

FIG. 3 is a schematic diagram of the example fill/insert device mounted on a medicament delivery device, according to the first embodiment;

FIG. 4 is a schematic diagram of the example fill/insert device after a fill driver has been released, according to the first embodiment;

FIG. 5 is a schematic diagram of the example fill/insert device after an insert driver has been released, according to the first embodiment;

FIGS. 6A-C are schematic, cross-sectional side views showing an example mechanism for automatically retracting a needle from a patient, according to various embodiments;

FIG. 7 is a schematic diagram of the example fill/insert device removed from the medicament delivery device after the filling and inserting steps have been performed, according to the first embodiment;

FIG. 8 is a schematic diagram of another example fill/insert device before being loaded with a medicament vial, according to a second embodiment;

FIG. 9 is a schematic diagram of the example fill/insert device after being loaded with a medicament vial, according to the second embodiment;

FIG. 10 is a schematic diagram of the example fill/insert device mounted on a medicament delivery device, according to the second embodiment;

FIG. 11 is a schematic diagram of the example fill/insert device after an insert driver and fill driver have been released, according to the second embodiment;

FIG. 12 is a schematic diagram of the example fill/insert device removed from the medicament delivery device after the filling and inserting steps have been performed, according to the second embodiment;

FIG. 13 is a schematic diagram of another example fill/insert device before being loaded with a medicament vial, according to a third embodiment;

FIG. 14 is a schematic diagram of the example fill/insert device after being loaded with a medicament vial, according to the third embodiment;

FIG. 15 is a schematic diagram of the example fill/insert device after being loaded through twisting of a knob, according to the third embodiment;

FIG. 16 is a schematic diagram of the loaded example fill/insert device mounted on a medicament delivery device, according to the third embodiment;

FIG. 17 is a schematic diagram of the example fill/insert device after an insert driver has been released, according to the third embodiment;

FIG. 18 is a schematic diagram of the example fill/insert device after a fill driver has been released, according to the third embodiment;

FIG. 19 is a schematic diagram of the example fill/insert device depicting an alternative technique for releasing the fill driver, according to the third embodiment;

FIG. 20 is a schematic diagram of another example fill/insert device, preloaded with medicament, before being mounted to a medicament delivery device, according to a fourth embodiment;

FIG. 21 is a schematic diagram of the example fill/insert device after being mounted to the medicament delivery device, according to the fourth embodiment;

FIG. 22 is a schematic diagram of the example fill/insert device after a fill driver has been driven and an insert driver has been loaded, according to the fourth embodiment;

FIG. 23 is a schematic diagram of the example fill/insert device after the insert driver has been released, according to the fourth embodiment; and

FIG. 24 is a schematic diagram of the example fill/insert device removed from the medicament delivery device after the filling and inserting steps have been performed, according to the fourth embodiment.

### DETAILED DESCRIPTION

Certain embodiments of the present invention feature a fill/insert device that can detachably mount to an infusion-type medicament delivery device (e.g., a patch pump). Example patch pump devices that can be used with the invention are described in U.S. Pat. Nos. 8,672,873, 8,547,239, 8,945,064 and 9,211,378, the contents of each of which are incorporated by reference as if set forth herein in their entireties. The fill/insert device can act to both fill the patch pump with medicament or other fluid and insert a cannula into the subcutaneous tissue of a patient, upon a single action by the patient (or in some cases, a limited number of interrelated actions, as compared to conventional devices). Although this application will often refer to actions taken by a “patient,” it should be understood that any such actions can also be performed by any “user” (e.g., a caregiver or other third party).

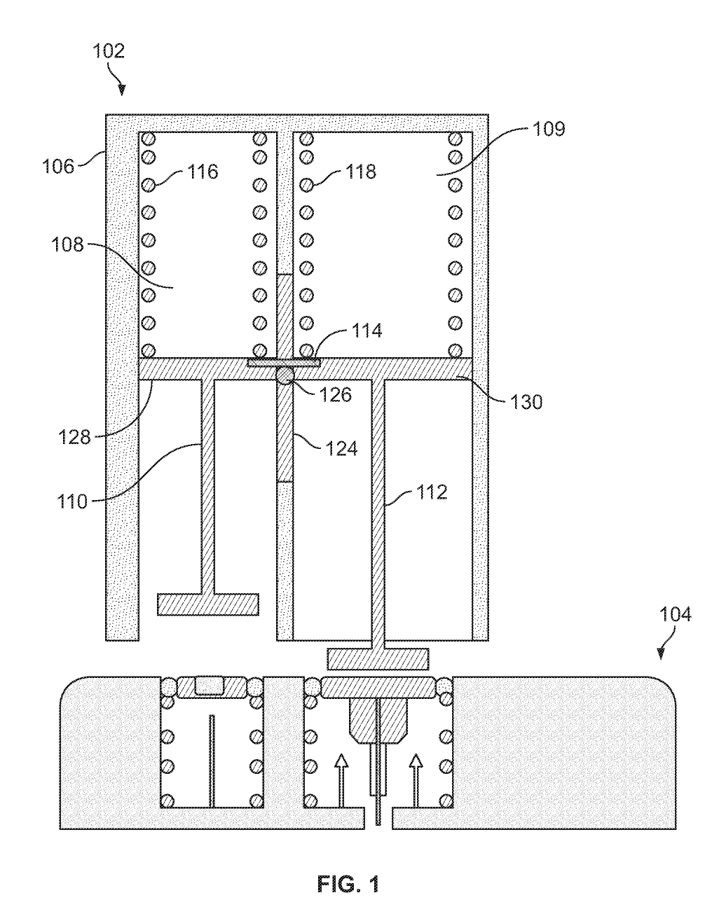

FIG. 1 shows an example fill/insert device 102 that can detachably mount to a medicament delivery device 104. The fill/insert device 102 can include an outer housing 106 having two internal chambers 108, 109. The first chamber 108 can house a fill driver 110 and the second chamber 109 can house an insert driver 112. As described in more detail below, the fill driver 110 and insert driver 112 are used to direct medicament into the delivery device 104 and a cannula into a patient's subcutaneous tissue, respectively. In general, the fill driver 110 and insert driver 112 can include any mechanism capable of performing these acts. For example, as shown in FIG. 1, the fill driver 110 and insert driver 112 can include piston-shaped shaped plungers attached to the housing 106 via drive springs 116, 118. In various other instances, the drivers 110, 112 can (i) take different shapes (e.g., solid cylinder, cylindrical shell, etc.), (ii) be driven differently (e.g., via hydraulic, pneumatic, electronic actuators, etc.), and (iii) include non-plunger type mechanisms. The fill driver 110 and insert driver 112 can be of the same mechanism type or different mechanism types (as shown with respect to other embodiments described below). As shown, the fill driver 110 and insert driver 112 may be attached to one another via an interlink 114 to correlate or coordinate actuation.

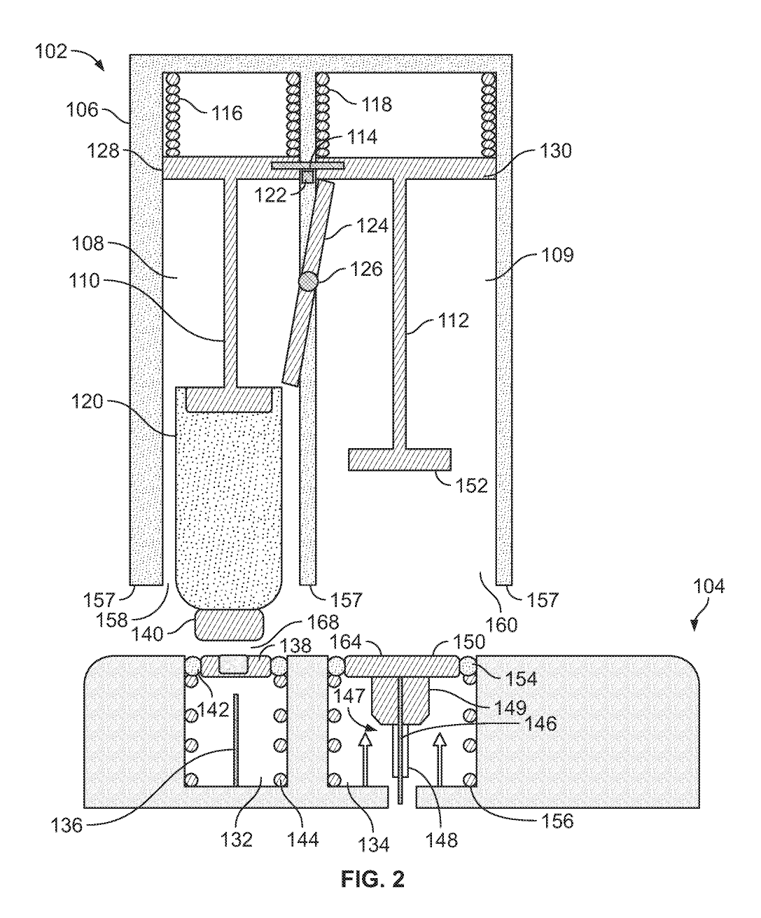

As shown, for example, in FIG. 2, the fill/insert device 102 is adapted to accept a medicament supply, e.g., a medicament vial 120. In other embodiments, the medicament supply may be stored in a different container type; for example, a squeeze tube, a burstable packet, etc. In some cases, the fill/insert device 102 may be provided to the patient pre-loaded with medicament. In other cases, the fill/insert device 102 is provided to the patient not loaded with medicament. In such cases, the patient can load the device 102 with medicament, which may include attaching the medicament vial 120 to fill driver 110. During the act of loading the medicament vial 120 (or, in some cases, afterwards) the patient can apply a compressive or linear force to the fill driver 110 (e.g., indirectly through a direct compressive or linear force applied to the medicament vial 120), which can result in a compression/loading of drive spring 116. In embodiments in which the fill driver 110 and insert driver 112 are attached via the interlink 114, the force that loads the drive spring 116 can also load the insert driver 112 and drive spring 118. In other embodiments, the insert driver 112 and drive spring 118 can be compressed/loaded separately from the fill driver 110 and drive spring 116. For example, the insert drive 112 and drive spring 118 can be preloaded before the device 102 is provided to the patient or the patient can load these components in a separate step.

In general, drive springs 116, 118 can be locked in a compressed state using any known technique. For example, in the embodiment shown in FIG. 2, the loading force may force interlink 114 over a displaceable catch 122 that moves under the loading force to allow the interlink 114 to pass, but does not move against the force applied by drive springs 116, 118 and holds the springs 116, 118 in a loaded position. Many other techniques for locking the springs are possible (e.g., mechanical catches adapted to hold either of the drivers 110, 112 as opposed to the interlink 114).

In some embodiments, the driving action of either the fill driver 110 or the insert driver 112 may be subject to an additional restriction to which the other driver is not subject. For example, as shown in FIGS. 1-5, the fill/insert device 102 can include a pivot bar 124 adapted to pivot about pivot point 126. In some instances, when the drivers 110, 112 are in an unloaded position (see FIG. 1), the pivot bar 124 presents no restriction to the loading of the drivers 110, 112, e.g., upper portions 128, 130 of the drivers 110, 112 can hold the pivot bar 124 in a vertical orientation as the drivers 110, 112 translate upwards within the housing 106. Device 102 can be configured such that when drivers 110, 112 are translated enough to compress drive springs 116, 118 into a loaded configuration (e.g., when the interlink 114 surpasses catch 122), upper portions 128, 130 can be in a position such that they no longer restrict the motion of pivot bar 124. At this point, pivot bar 124 can rotate clockwise such that it provides an additional restriction to the driving action of insert driver 112, to which fill driver 110 is not subject. Pivot bar 124 can be adapted to pivot under many different forces. As a few non-limiting examples, the bar 124 can be biased to rotate (e.g., via action of a torsional spring), or can be pushed by contact with the upwardly-translating medicament vial 120. In other embodiments, the device 102 can be configured such that the pivot bar 124 applies an additional restriction to the fill driver 110, rather than the fill driver 112 (e.g., in an embodiment in which the insert driver 112 is released before the fill driver 110, described below).

As mentioned, the insert/fill device 102 interfaces with a medicament delivery device 104, e.g., a patch pump adapted to be adhered to a patient's skin in order to subcutaneously deliver medicament to the patient. In certain embodiments, the medicament delivery device 104 can feature a medicament fill port 132 and a cannula delivery port 134. In general, the medicament fill port 132 can include any structure capable of interfacing with the medicament supply contained in the insert/fill device 102 to deliver medicament to an internal reservoir of delivery device 104. As one example, the fill port 132 can include a hollow fill needle 136, a fill interface 138 adapted to interact with a dispensing portion 140 of the medicament vial 120, a seal 142 between the fill interface 138 and the fill port 132, and a fill reservoir spring 144. The fill interface 138 can be biased away from the hollow fill needle 136 contained in the delivery device 104. This bias can reduce the likelihood of inadvertent contact with the hollow fill needle 136.

Similarly, in general, the cannula delivery port 134 can include any structure capable of interfacing with the insert driver 112 to insert a cannula into the patient. As one example, the cannula delivery port 134 can include a needle 146 mated with a cannula assembly 147 (which may include a cannula 148 and a base 149), an insert interface 150 adapted to interact with a drive portion 152 of the insert driver 112, a seal 154 between the insert interface 150 and the cannula delivery port 134, and a cannula reservoir spring 156. In various embodiments, any or all of the structure described above for the example fill port 132 and cannula delivery port 134 may be excluded or substituted.

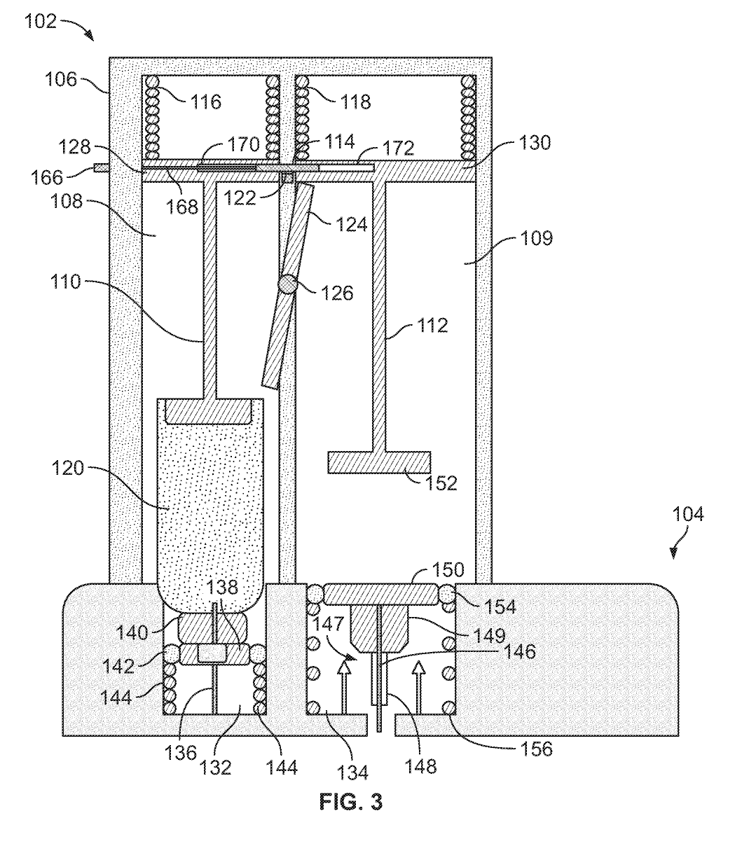

In various embodiments, when the fill/insert device 102 mounts to the medicament delivery device 104, an interface 157 mates with the delivery device 104. For example, outlets 158, 160 of housing chambers 108, 109, can align with inlets 162, 164 of fill port 132 and cannula delivery port 134, respectively. FIG. 3 shows an example illustration of the fill/insert device 102 mounted to the medicament delivery device 104. In some instances, the mounting can occur by the patient attaching the fill/insert device 102 to the delivery device 104 (which in some cases may already be adhered to the patient's skin) using known techniques, e.g., directing a lip on the housing 106 into a corresponding groove in the delivery device 104. Many other attachment techniques are possible (e.g., torsional bayonet-style retention tabs). Upon attachment (or in some cases, as the act of attachment), the dispensing portion 140 of the medicament vial 120 can interact with the fill interface 138, such that the hollow fill needle 136 is fluidically connected with the medicament supply (e.g., guided into the interior of the medicament vial 120). In some embodiments, the user applied force to attach the fill/insert device 102 to the delivery device 104 can compress the fill reservoir spring 144 to allow for the fluidic connection to occur.

In various embodiments, once the fill/insert device 102 is mounted to the delivery device 104, the system is ready for the patient to activate the medicament filling and cannula inserting steps. As mentioned, in some instances, both the filling step and the inserting step can be completed upon a single activation of a single trigger by the patient. In general, the trigger can be any action that results in the fill driver 110 (or, in some cases, the insert driver 112) being released. As one example, consider an embodiment in which the interlink 114 slidably rests within slots 170, 172 of upper portions 128, 130 of drivers 110, 112, respectively. In such an embodiment, the trigger may include a trigger button 166 connected to a push shaft 168 abutting the interlink 114. A patient's pushing of the trigger button 166 can cause the push shaft 168 to force the interlink 114 fully out of groove 170 into groove 172, thus decoupling the fill driver 110 from the interlink 114 and removing the resistance force generated by the interlink's interaction with the catch 122. As another example, the trigger button 166 may be a magnetic switch that creates a magnetic field that either attracts the interlink 114 into slot 170 or repels it into slot 172. Depending on how the drive spring 116 is held in a locked position, many other techniques for releasing the drive spring 116 are possible.

Regardless of how the drive spring 116 is released, upon release the drive spring 116 is free to expand and drive the fill driver 110 towards the delivery device 104. In the depicted embodiment (see FIG. 3), this results in piston portion 169 of the fill driver 110 being driven into a cylinder formed by the medicament vial 120, resulting in passage of the medicament contained therein through the hollow fill needle 136 into an internal reservoir of the delivery device 104. As shown in FIG. 3, in some cases, release of the fill driver 110 does not immediately result in release of the insert driver 112, because even when the resistance force of the interlink 114 is removed, the additional resistance force applied by the pivot bar 124 remains.

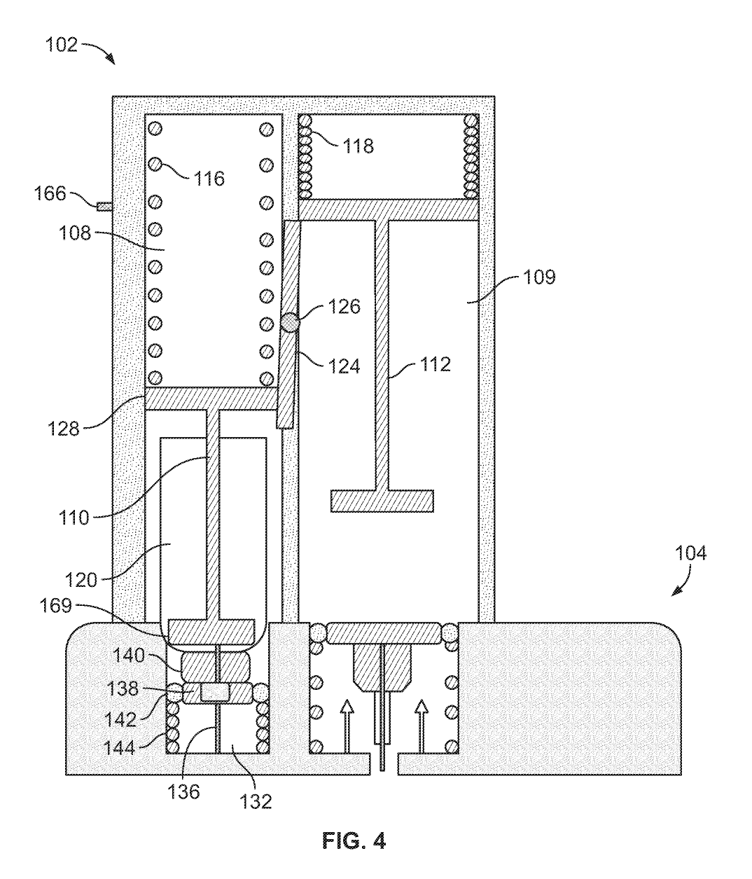

In some embodiments, the action of the released fill driver 110 will automatically cause the release of the insert driver 112. For example, in the depicted embodiment (see FIGS. 3-4), upon translating towards the delivery device 104, the upper portion 128 of the fill driver 110 may contact the pivot bar 124 and apply a moment to rotate the pivot bar 124 counterclockwise about pivot point 126 until the insert driver 112 is free to move. In some instances, the fill/insert device 102 can be configured such that the resistance force from the pivot bar 124 is not fully removed until the fill driver 110 reaches the bottom of its stroke and the filling step is substantially completed. In such instances, although both the filling and inserting steps are completed upon a single trigger by the patient, the steps are performed sequentially within the device 102. In other instances, the device 102 can be configured such that the resistance force from the pivot bar 124 is removed (and the inserting step commenced) at any point before the fill driver 110 reaches the end of its stroke (e.g., at the beginning of stroke).

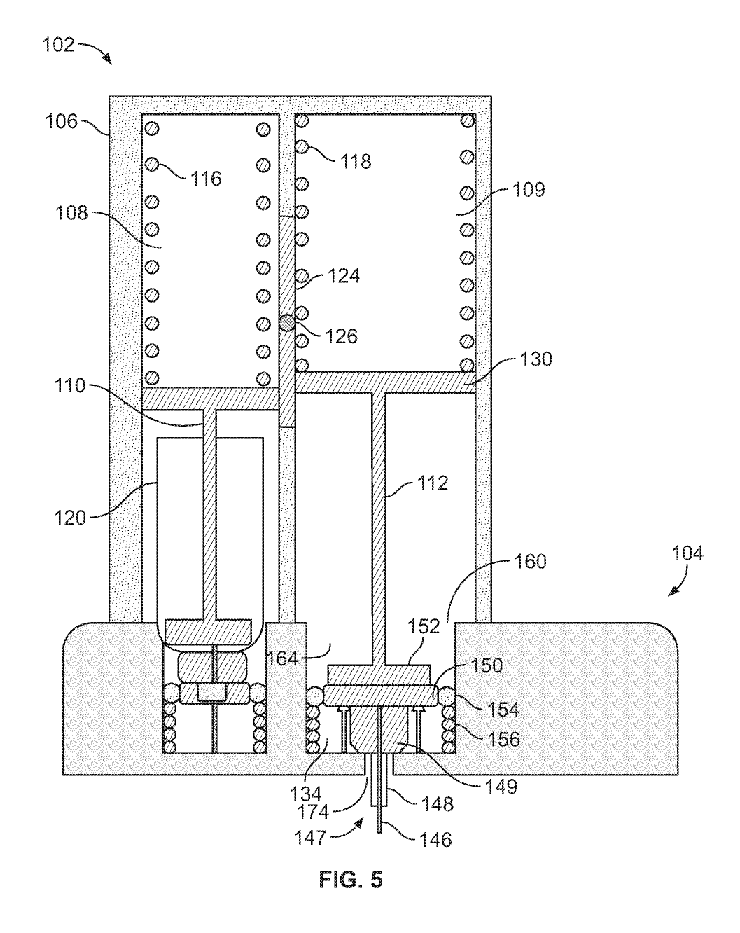

Absent the resistance force from either the interlink 114 or the pivot bar 124, drive spring 118 can expand, driving the insert driver 112 towards the delivery device 104. As shown, for example, in FIG. 5, release of the insert driver 112 can cause drive portion 152 to engage insert interface 150 within the cannula delivery port 134. In some instances, the insert interface 150 can be releasably coupled to the cannula assembly 147. In some cases, the needle 146 is attached to the insert interface 150 and fits through a lumen of the cannula 148. The force applied by the drive portion 152 can drive the insert interface 150, cannula assembly 147, and needle 146 downward, such that the needle 146 and cannula 148 exit a cannula outlet 174 in the base of the delivery device 104 and extend subcutaneously into the patient. The needle 146 can act to initially pierce the skin/tissue and to carry the cannula 148 to a desired location. At the bottom of the insert driver's stroke, the base 149 can be seated at a bottom of the cannula delivery port 134.

In some embodiments, the base 149 defines an internal chamber in fluidic communication with the lumen of the cannula 148. Upon being seated, the base 149 can automatically align with a medicament delivery channel of the delivery device 104 fed by the internal medicament reservoir, such that the lumen is in fluidic communication with the delivery channel. More details on alignment of the base 149 to fluidically couple the cannula 148 with the delivery device 104 are contained in U.S. Pat. No. 9,005,169, the contents of which are incorporated by reference as if set forth herein in its entirety.

In certain embodiments, once the cannula 148 is placed within the patient, the needle 146 can be automatically retracted out of the patient. There are many ways this can be accomplished. As one example, the cannula delivery port 134 can include a holding structure (e.g., compliant arms) that grabs the cannula base 149 upon being seated in the bottom of the delivery port 134. At the bottom of the insert driver's stroke, the drive spring 118 can be decoupled from the insert driver 112 using conventional known techniques. Upon decoupling, the drive force applied by the drive spring 118 is removed from the cannula reservoir spring 156, which can then expand to force the insert interface 150 upwards (sometimes breaking a frangible connection between the interface 150 and the base 149), resulting in withdrawal of the needle 146 from the patient. In this example, because the base 149 is held in place, neither the base 149 nor the cannula 148 retract with the interface 150 and remain in place to deliver medicament to the patient.

In other embodiments, the structure contained in the delivery port 134 can be different to accomplish automatic needle retraction differently. Example techniques for the automatic retraction of a needle are contained in U.S. Pat. No. 7,846,132, the contents of which are incorporated by reference as if set forth herein in its entirety (see, e.g., FIGS. 1-3, 5, 6-7, and 9-13). In various embodiments, the delivery port 134 can be modified to contain the structure taught by the '132 patent (or similar structure) for automatic retraction of a needle.

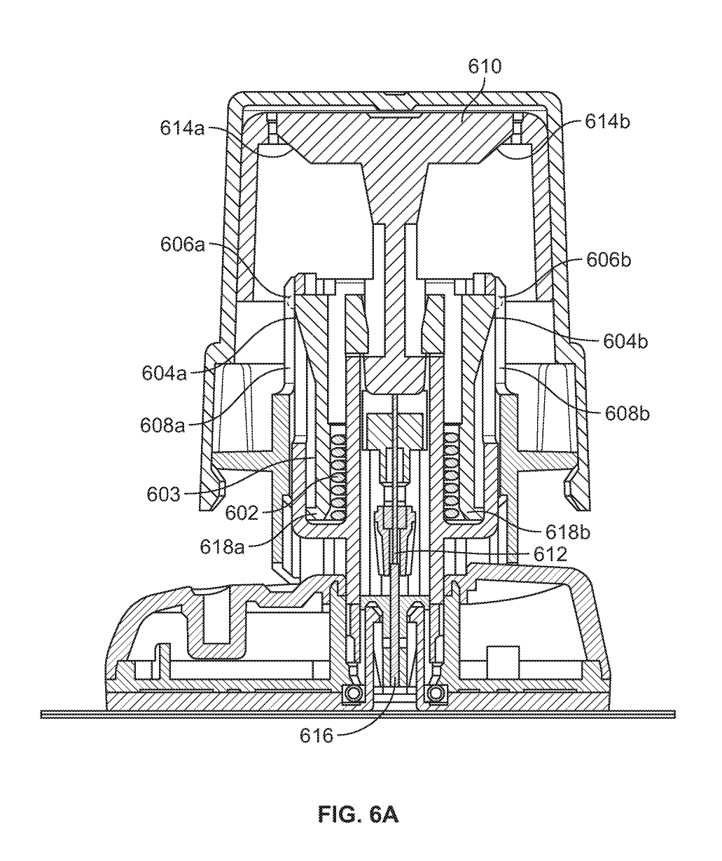

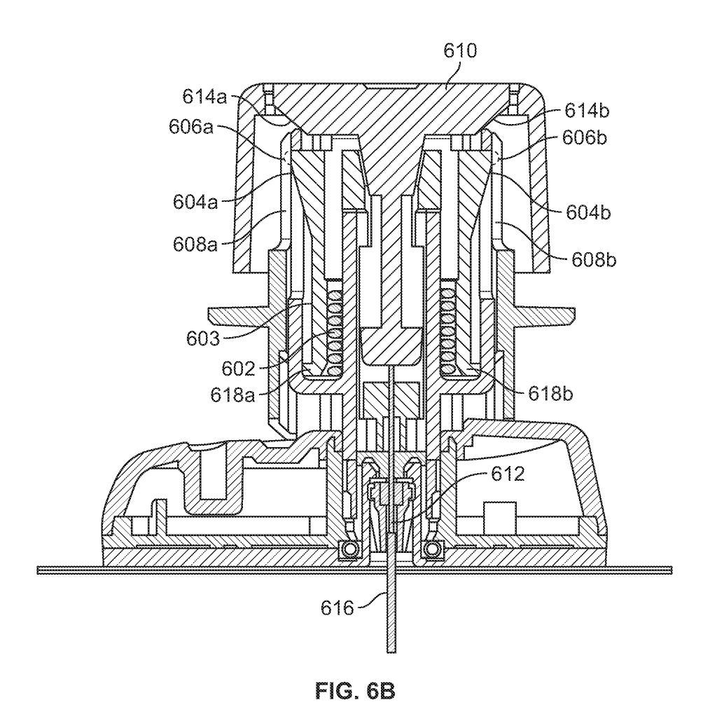

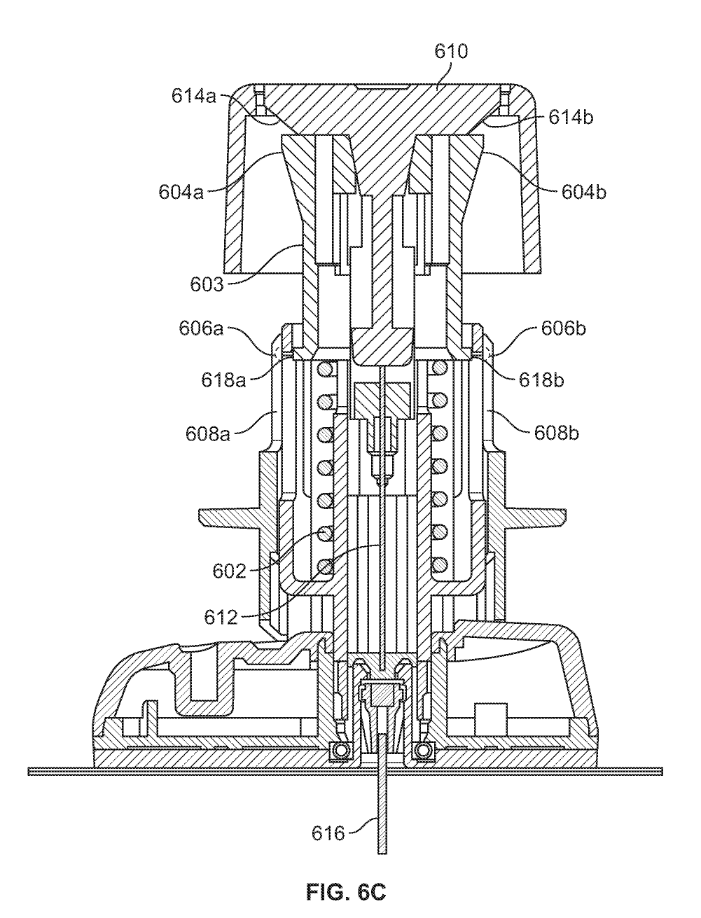

Another example technique for automatic retraction of a needle is shown in FIGS. 6A-C. In this technique, a retraction spring 602 can be held in a loaded position by a shuttle 603 having two protruding surfaces 604a, 604b that fit into corresponding notches 606a, 606b in tensioned bars 608a, 608b. A needle driver 610 carrying a needle 612 coupled to a cannula 616 can be driven downwards to insert the needle 612 and the cannula 616 into a patient. The needle driver 610 can feature two inclined surfaces 614a, 614b that, once the needle 612 and cannula 616 have been driven a sufficient distance (e.g., such that the cannula 616 is placed at a desirable location within the patient), can engage tensioned bars 608a, 608b, pushing them outwards such that protruding surfaces 604a, 604b are released from notches 606a, 606b (see FIG. 6B). Once the protruding surfaces are released, the force holding the retraction spring 602 in place is removed and the retraction spring 602 can expand, pushing the shuttle 603 and the needle driver 610 upwards, thereby withdrawing the needle 612 from the patient, while leaving the cannula 616 in place. In some cases, after the protruding surfaces 606a, 606b have translated upwards beyond the tensioned bars 608a, 608b, the tensioned bars 608a, 608b can return to their original position. In some such cases, after the needle driver 610 has been retracted a desirable distance, notches 606a, 606b can engage nubs 618a, 618b of the shuttle 603 to prevent further retraction (see FIG. 6C). In various embodiments, the delivery port 134 can be modified to contain the structure used by this example technique (or similar techniques) for automatic retraction of a needle. In other embodiments, the needle 146 may not be automatically retracted at all, but rather be retracted manually and/or upon the patient's manual removal of the fill/insert device 102 from the delivery device 104.

In various other implementations of this example embodiment, the fill/insert device 102 can be configured such that the insert driver 112 is released before the fill driver 110. As one example, as mentioned above, the pivot bar 124 can be biased to provide an additional resistance force to the fill driver 110, instead of the insert driver 112. In such instances, the interactions of the fill driver 110 and the insert driver 112 with the pivot bar 124 can be reversed, such that the insert driver 112 is released before the fill driver 110 and, therefore, the insert step is performed before the filling step.

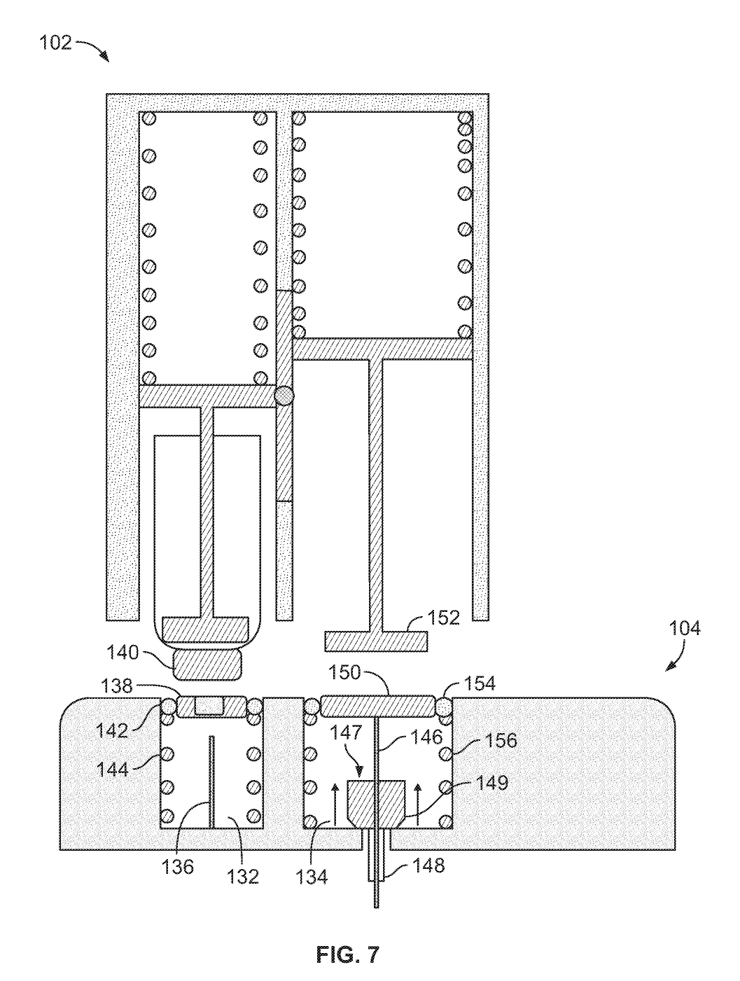

In various embodiments, regardless of whether the needle 146 is automatically retracted, the fill/insert device 102 will be removed from the delivery device 104 after completion of the fill and insert steps. The user can manually detach the fill/insert device 102 from the delivery device 104 (e.g., disengage a lip from a corresponding groove, etc.). In some instances, fill reservoir spring 144 and cannula reservoir spring 156 may expand as the fill/insert device 102 is removed, which can provide a guiding force to ensure the device 102 does not get stuck upon removal. In some such instances, fill interface 138 and insert interface 150, and associated seals 142, 154, can create an IPX8 waterproof seal. FIG. 7 is a schematic diagram showing the fill/insert device 102 after it has been removed from the delivery device 104. As depicted, in certain embodiments, the hollow fill needle 136 and needle 146 can remain in the delivery device 104 after the fill/insert device 102 is removed. For example, the hollow fill needle 136 may remain attached to the delivery device 104 and the needle 146 may remain attached to the insert interface 150. In other embodiments, some of which are detailed below, either or both of the needles 136, 146 can be removed from the delivery device 104 along with the fill/insert device 102. In the embodiment depicted in FIG. 7, upon removal of the fill/insert device 102, the fill interface 138 and insert interface 150 can return to a top surface of the delivery device 104. In such embodiments, the interfaces 138, 150 may be biased to prevent inadvertent contact with the needles 136, 146.

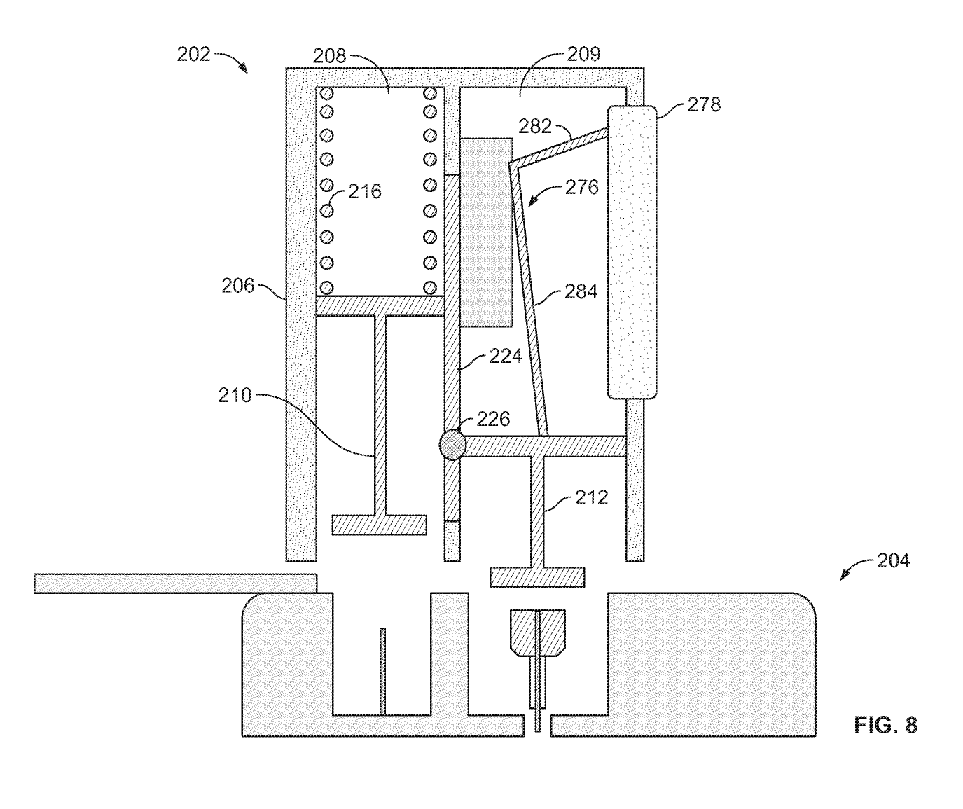

A second example embodiment is depicted in FIGS. 8-12. As shown in FIG. 8, a fill/insert device 202 can detachably mount to a medicament delivery device 204. The fill/insert device 202 can include an outer housing 206 having two internal chambers 208, 209. The first chamber 208 can house a fill driver 210 and the second chamber 209 can house an insert driver 212 connected by a lever system 276 to a button 278. The lever system 276 can include a first arm 282 and a second arm 284. The button 278 may be pushed by the user to release the insert driver 212 which, as described below, can also result in release of the fill driver 210.

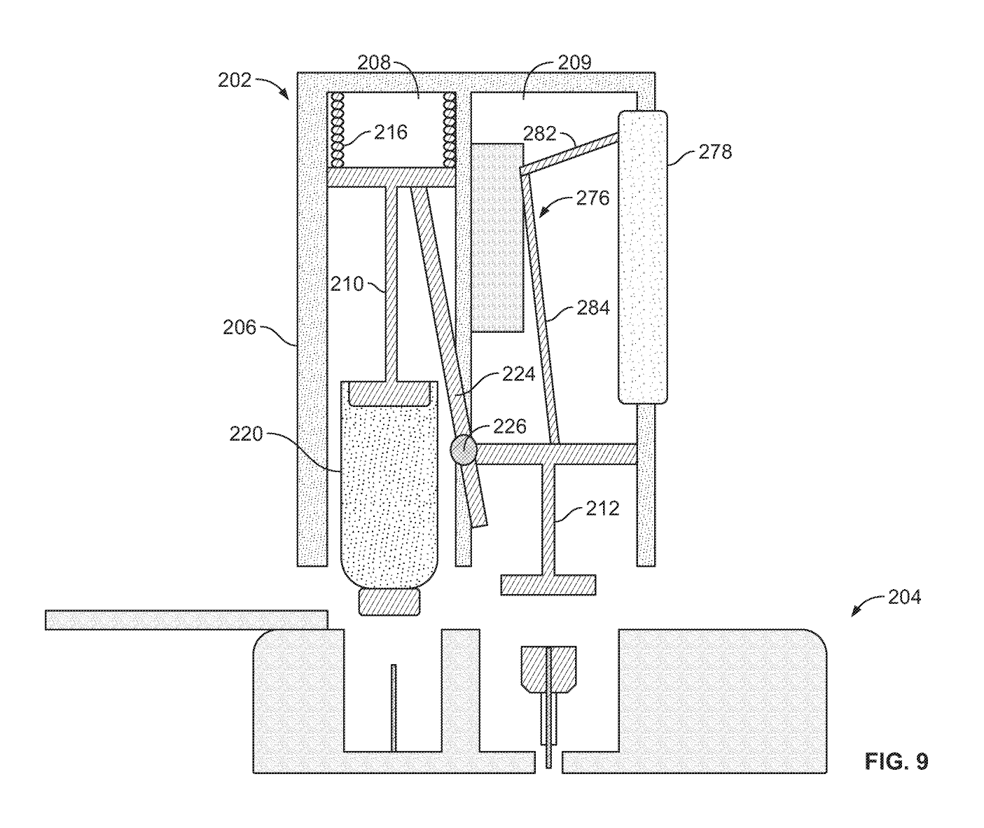

As described with respect to the first example embodiment, the fill/insert device 202 shown in FIG. 8 is adapted to accept a medicament vial 220 (see FIG. 9). As described, in some cases, loading of the medicament vial 220 can result in compression/loading of a drive spring 216. When the drive spring 216 is fully loaded, a pivot bar 224 may rotate about a pivot point 226 to hold the fill driver 210 in a loaded position. In some embodiments, as shown, for example, in FIG. 8, the insert driver 212 is in a loaded position prior to the vial 220 being loaded (e.g., at the time the device 102 is provided to the patient).

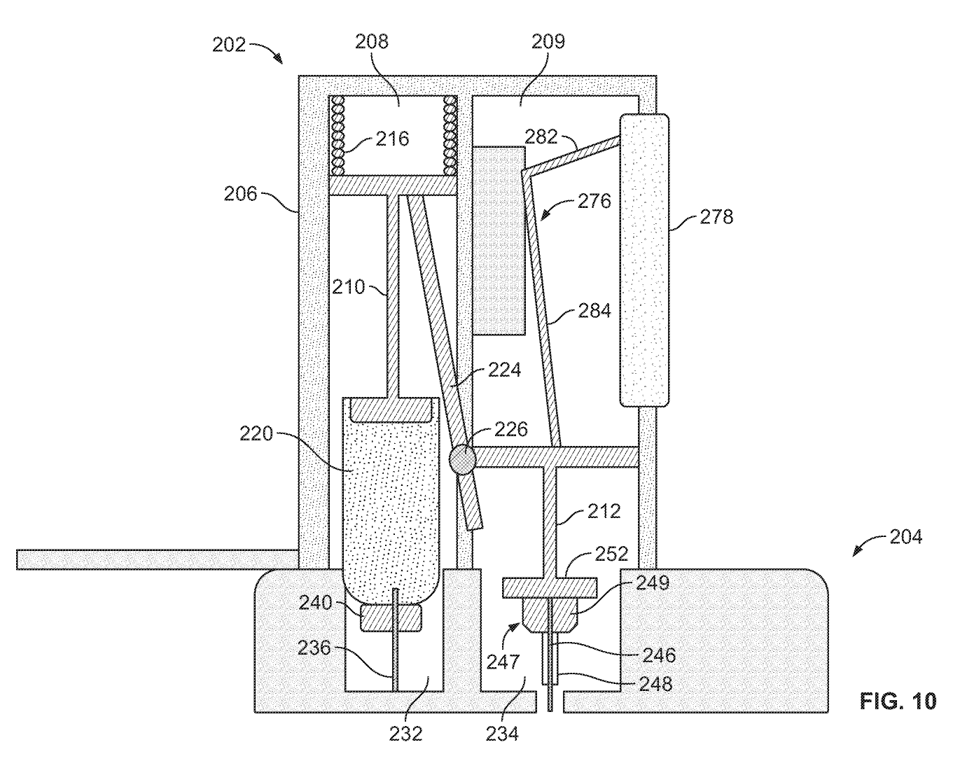

FIG. 10 schematically depicts the fill/insert device 202 mounted to the medicament delivery device 204. As previously described, the medicament delivery device 204 can include a medicament fill port 232 and a cannula delivery port 234, adapted to interact with the fill driver 210 and insert driver 212, respectively. Upon attachment of the fill/insert device 202 to the delivery device 204, a drive portion 252 of the insert driver 212 may engage (or, in some cases, be positioned directly above) a base 249 of a cannula assembly 247. In addition to the base 249, the cannula assembly 247 can feature a cannula 248 attached to the base 249.

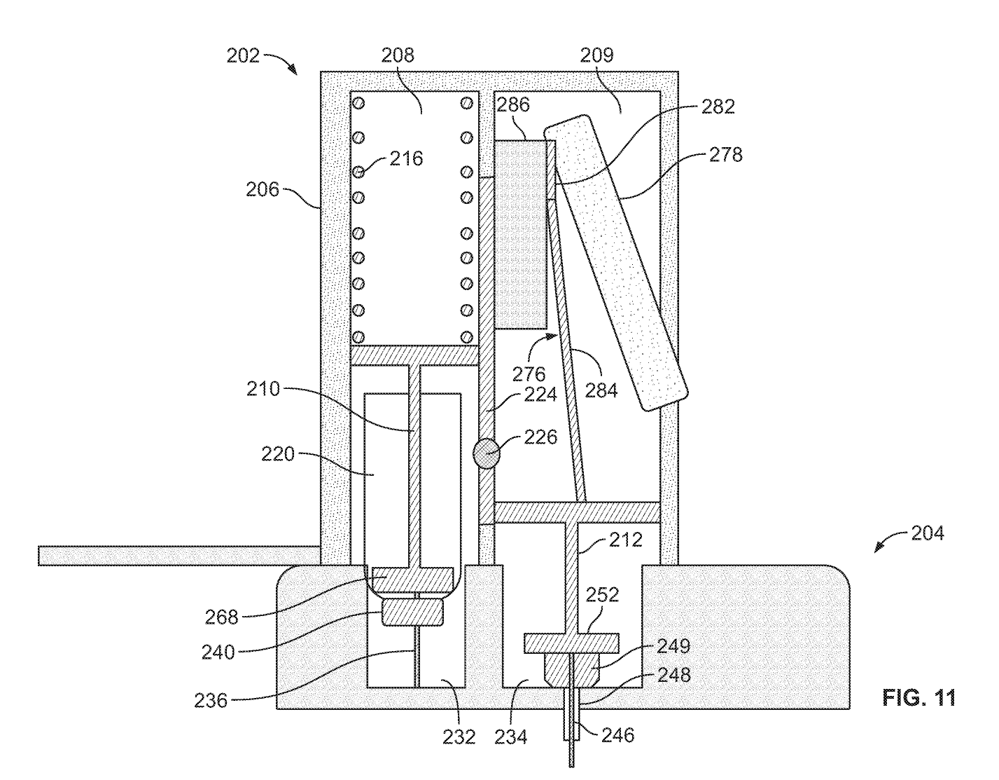

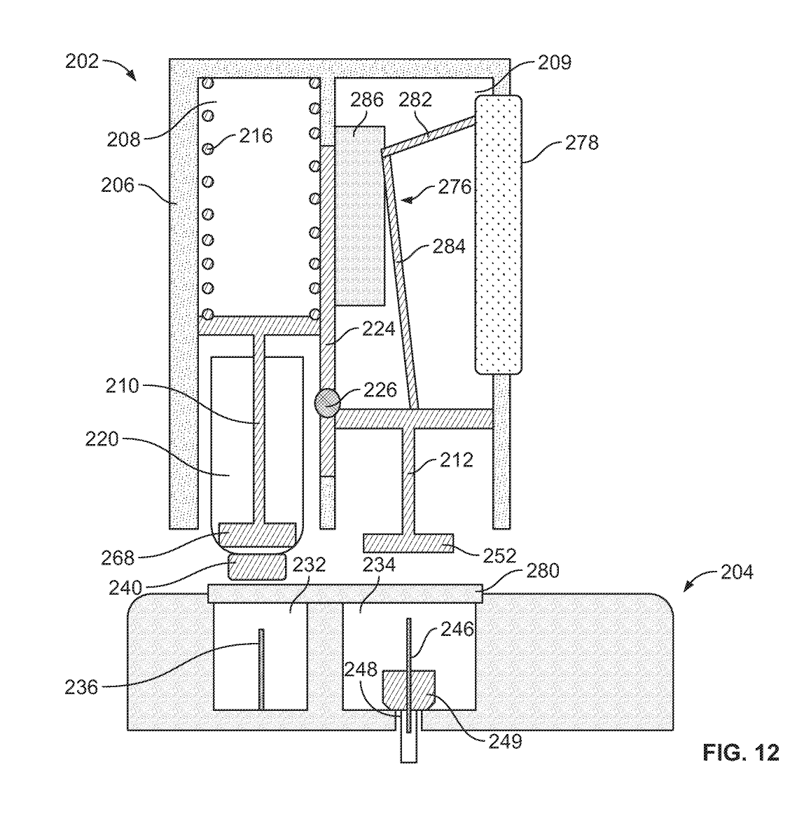

Once the fill/insert device 202 is mounted, button 278 can be pressed (see FIG. 11), which can cause lever system 276 to straighten. In some cases, straightening of lever system 276 can result from the first lever arm 282 being forced to rotate with respect to the second lever arm 284. Such rotation can be caused by the moment applied from the pressed button 278. In some cases (as shown) the moment is generated about a bumper 286. Straightening of the lever system 276 can force the insert driver 212 downwards towards the delivery port 234 of the delivery device 204. As described above with respect to the first example embodiment, translation of the insert driver 212 downwards can result in placement of the cannula 248 in the patient. Once the button 278 is released, the lever system 276 may return to its original position (see FIG. 12), such that the fill/insert device 202 can be re-used with another (or, in some cases, the same) delivery device 204.

In various embodiments, translation of the insert driver 212 downwards can also result in triggering of the filling step. For example, as shown in FIG. 11, translation of the insert driver 212 downwards can cause the pivot bar 224 to rotate such that the drive spring 216 is released. Once released, the drive spring 216 can drive the fill driver 210 towards the delivery device 204. In some cases, a plunger 268 can force medicament out of the medicament vial 220, through the fill needle 236, and into a reservoir in the interior of the delivery device 204.

In various other implementations of the second example embodiment, the fill/insert device 202 can be configured such that the fill driver 210 is released before the insert driver 212. As one example, the button 278 and lever system 276 can be configured to release the fill driver 210, which results in a subsequent rotation of the pivot bar 224, causing the insert driver 212 to release. In still other implementations, both the fill driver 210 and the insert driver 212 can be released by pressing a button attached to a lever system.

Regardless of how the drivers 210, 212 are released, once both the fill driver 210 and the insert driver 212 have reached the bottom of stroke and both the filling and inserting steps are completed, the fill/insert device 202 can be removed from the delivery device 204 (see FIG. 12). In some cases, the needle 246 is automatically retracted out of the patient's skin into the cannula deployment port 234 before the fill/insert device 202 is removed. The fill/insert device 202 can be removed from the delivery device 204 using any of the techniques previously described. As another example, the fill/insert device 202 can be removed by the action of a lid 280 translating over and closing off the top open surfaces of the fill port 232 and cannula delivery port 234. The lid 280 can create an IPX8 waterproof seal. In some instances, the lid 280 can be part of an indicator unit (described in, e.g., U.S. Pat. Nos. 8,672,873 and 8,547,237, incorporated by reference herein). In such instances, translation of the lid 280 can be caused by fully mating the indicator unit with the delivery device 204. In other instances, the lid 280 can be a standalone element. In such instances, the lid 280 can be closed using any conventional technique (e.g., via a spring loaded release or simply by a manual action of a user).

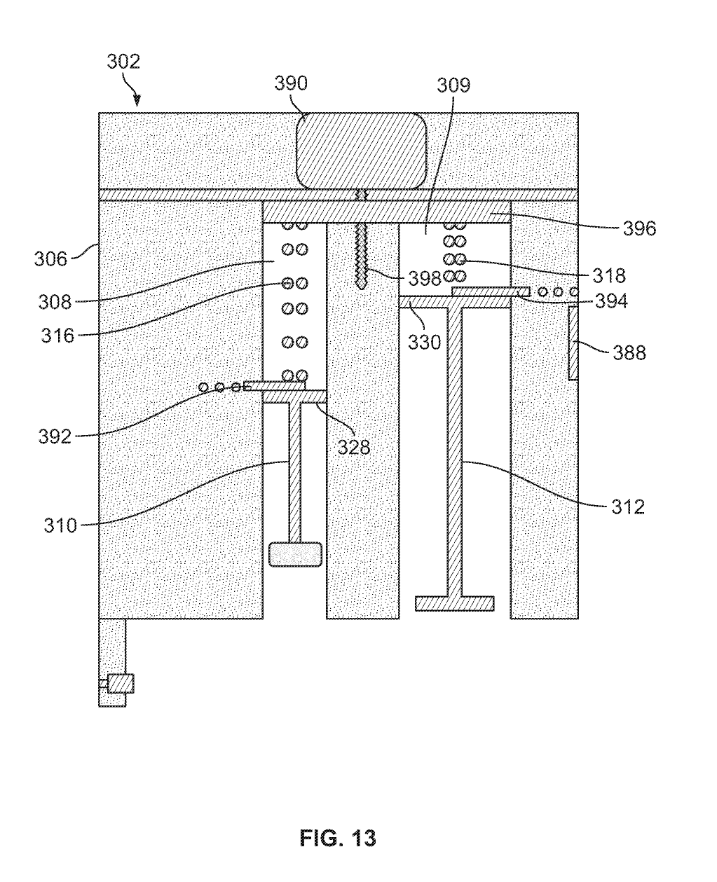

A third example embodiment is depicted in FIGS. 13-19. As shown in FIG. 13, in this embodiment the fill/insert device 302 can include an outer housing 306 that includes a retraction button 388 and a rotatable knob 390. As described in previous embodiments, a first chamber 308 can house a fill driver 310 and a second chamber 309 can house an insert driver 312. The upper portions 328 and 330 of the fill and insert drivers 310, 312, may be attached to retractable arms 392, 394, which are connected to the housing 306. The fill and insert drivers 310, 312 may also be coupled to a platform 396 via drive springs 316, 318, in some cases through a connection between the retractable arms 392, 394 and the platform 396. In certain instances, the drivers 310, 312 are suspended within chambers 308, 309 as a result of their attachment to retractable arms 392, 394. In some cases, the drivers 310, 312 and the retractable arms 392, 394 are attached via a frangible connection.

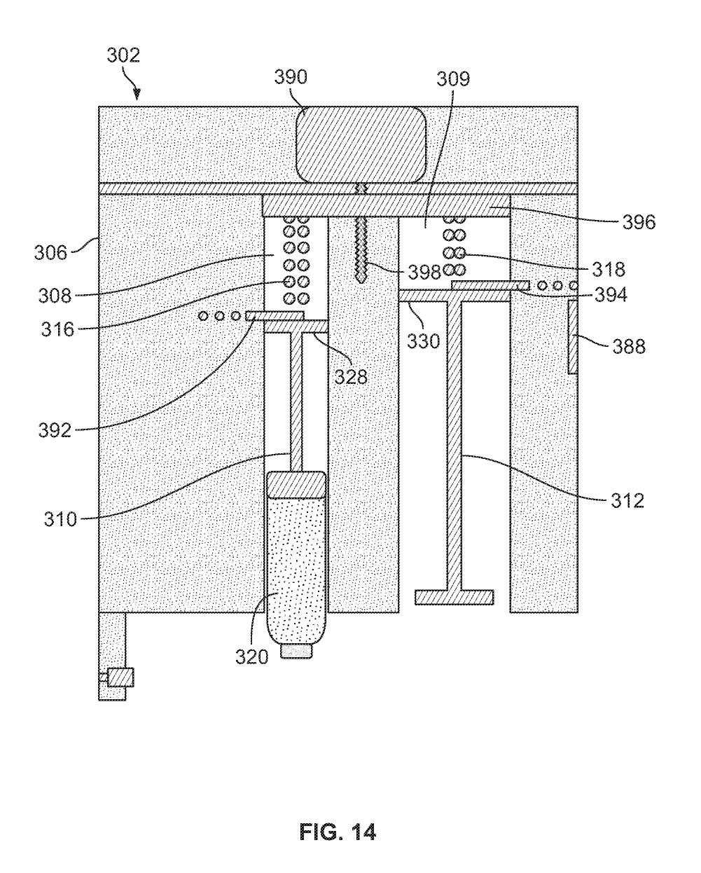

The fill/insert device 302 shown in FIG. 14 can accept a medicament vial 320 (see FIG. 14). During the act of loading the medicament vial 320 (or, in some cases, afterwards) a patient can apply a compressive force to the fill driver 310 (e.g., indirectly through a direct compressive force applied to the medicament vial 320), which can result in a compression/loading of drive spring 316. As described below, in some cases, the loading of the medicament vial may result in a partial compression/loading, as additional compression/loading can also occur.

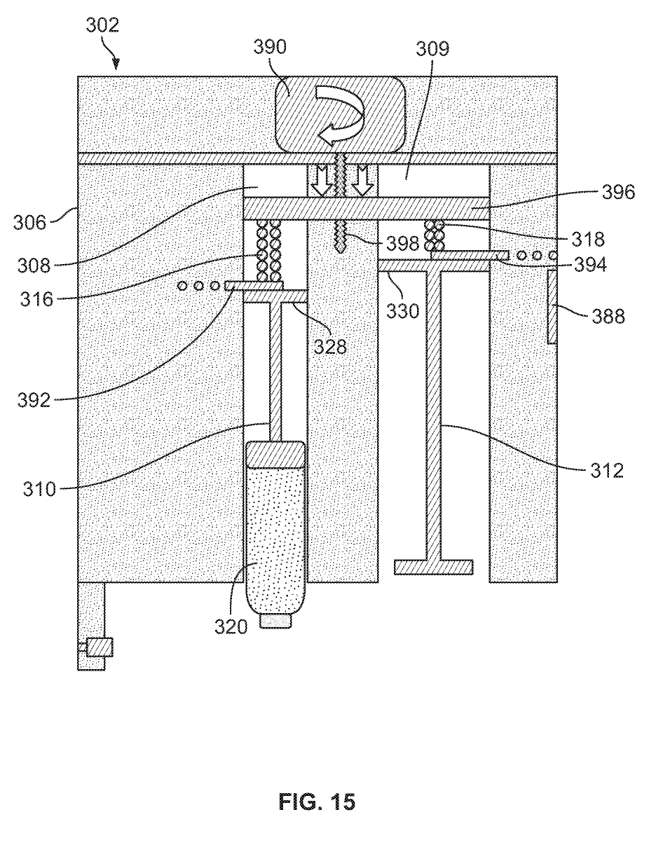

FIG. 15 is a schematic diagram showing an example technique for loading drive springs 316, 318. As shown, knob 390 may engage a lead screw 398 threaded into a threaded bore in platform 396. A user may turn the knob 390, which can drive the lead screw 398 resulting in the platform 396 translating towards the drivers 310, 312. Translation of the platform 396 can compress drive springs 316, 318 (in some cases further compressing drive spring 316) such that they are in a fully loaded position. The drive springs 316, 318 may be held in a loaded position using any technique described previously or any known conventional technique. For example, with reference to FIG. 15, the drive springs 316, 318 can be held in a loaded position between the platform 396 and the retractable arms 392, 394. A user can turn the knob 390 resulting in loading of the drive springs 316, 318 either before or after the fill/insert device 302 is attached to a delivery device 304.

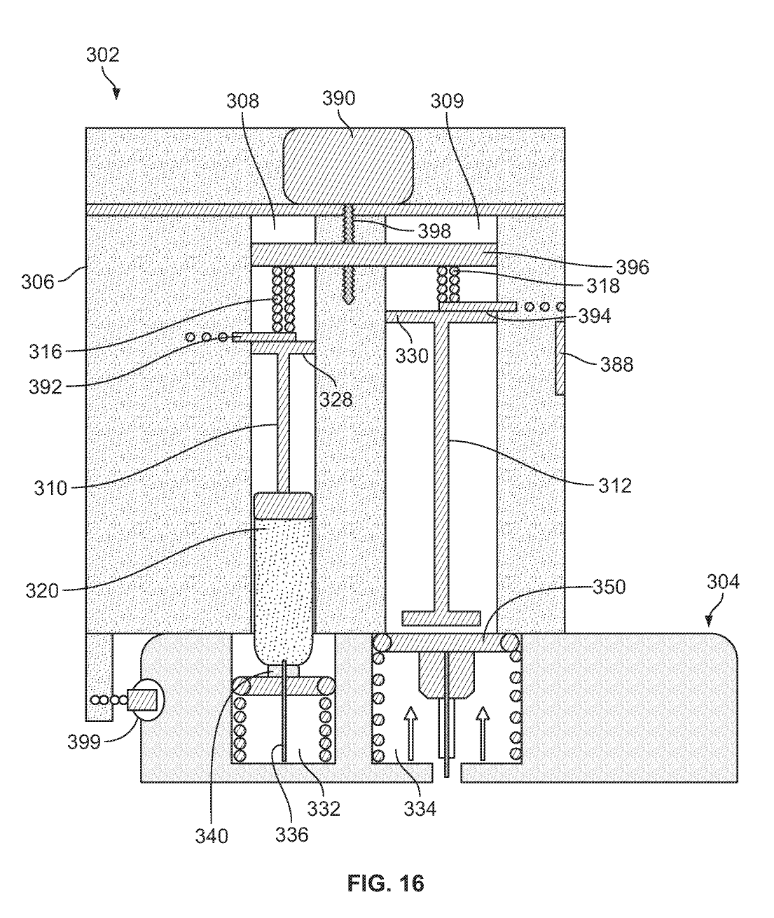

FIG. 16 is a schematic depiction of the fill/insert device 302 mounted to a medicament delivery device 304. In some cases, the fill/insert device 302 connects to the delivery device 304 via a spring loaded latch 399. In other embodiments, the connection can be accomplished using any other known technique (e.g. a magnetic force, compliant arms, loop and hook fasteners, etc.). When the fill/insert device 302 mounts to the medicament delivery device 304, the medicament vial 320 can align with the medicament fill port 332 and the insert driver 312 can align with the cannula delivery port 334.

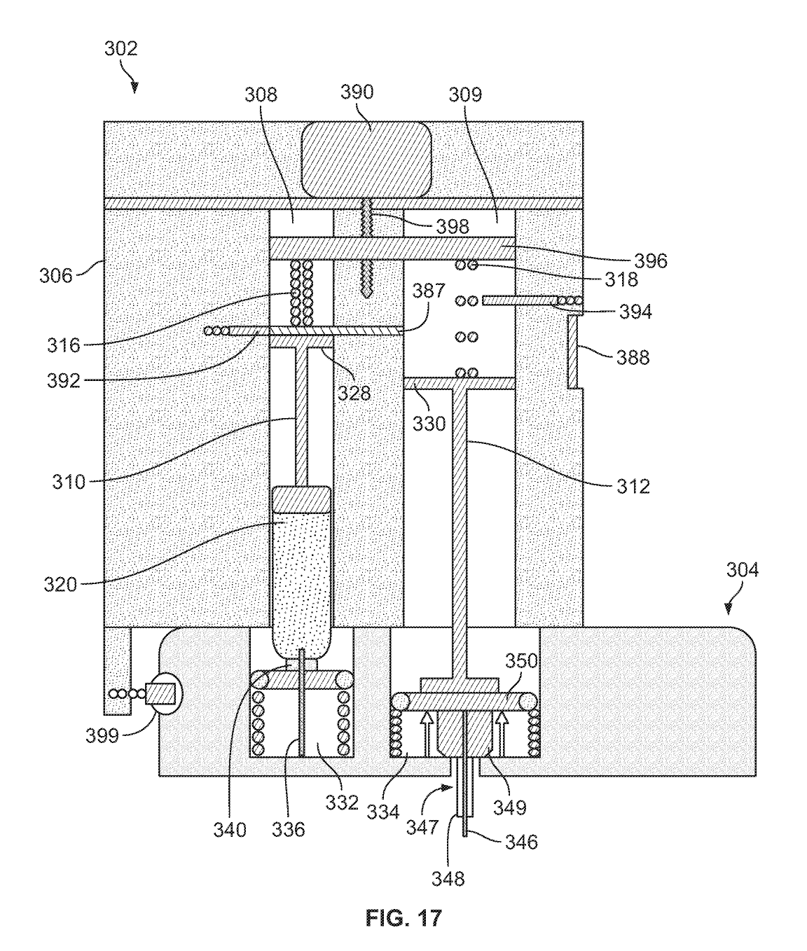

In various embodiments, after the fill/insert device 302 is mounted onto the delivery device 304, the drive spring 318 can be released. One example technique for releasing the drive spring 318 can include retracting retractable arm 394, such that it no longer impedes the expansion of the drive spring 318 (see FIG. 17). In some cases, retraction of the retractable arm 394 can be effected by a user pressing retraction button 388. In general, retractable arm 394 can be retracted using any known technique; for example, pressing the retraction button 388 may compress a spring or generate a magnetic field that results in retraction of the retraction arm 394. In some instances, retraction of the retraction arm 394 may result in the severing of the frangible connection between the retractable arm 394 and the insert driver 312. Once the drive spring 318 is released it can drive the insert driver 312, insert interface 350, needle 346, and cannula assembly 347 downwards, resulting in insertion of cannula 348 into a patient, as described with respect to previous embodiments. In some cases, after the cannula 348 is placed, the needle 346 is automatically retracted, as described above.

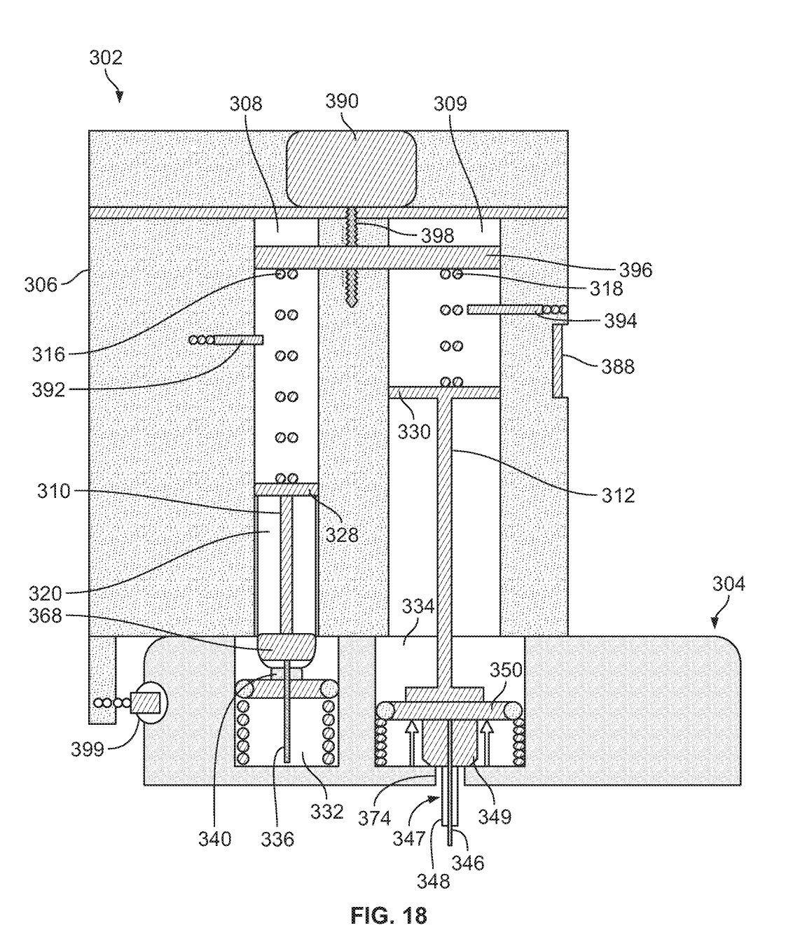

In some embodiments, as shown, for example, in FIGS. 17-18, release of the insert driver 312 can result in a release of the fill driver 310; for example, by retracting retraction arm 392. In general, this can be done using any conventional technique, including any of the techniques previously described herein. In some instances, retraction of retraction arm 392 is mechanical; for example, an upper portion 330 of the insert driver 312 can contact a retraction arm push shaft 387 which forces retraction arm 392 into a retracted position (see FIG. 17). In other instances, retraction of the retraction arm 392 can be caused by a magnetic field and/or electrical signal.

The fill/insert device 302 can be configured such that the fill driver 310 is released at any point along the stroke of the insert driver 312 (e.g., the end of stroke, the beginning of stroke, etc.). Once drive spring 316 is released it can drive a plunger portion 369 of the fill driver 310 through a cylinder of the medicament vial 320 to force medicament through a hollow fill needle 336 and into an internal reservoir of the delivery device 304, as described above with respect to other embodiments.

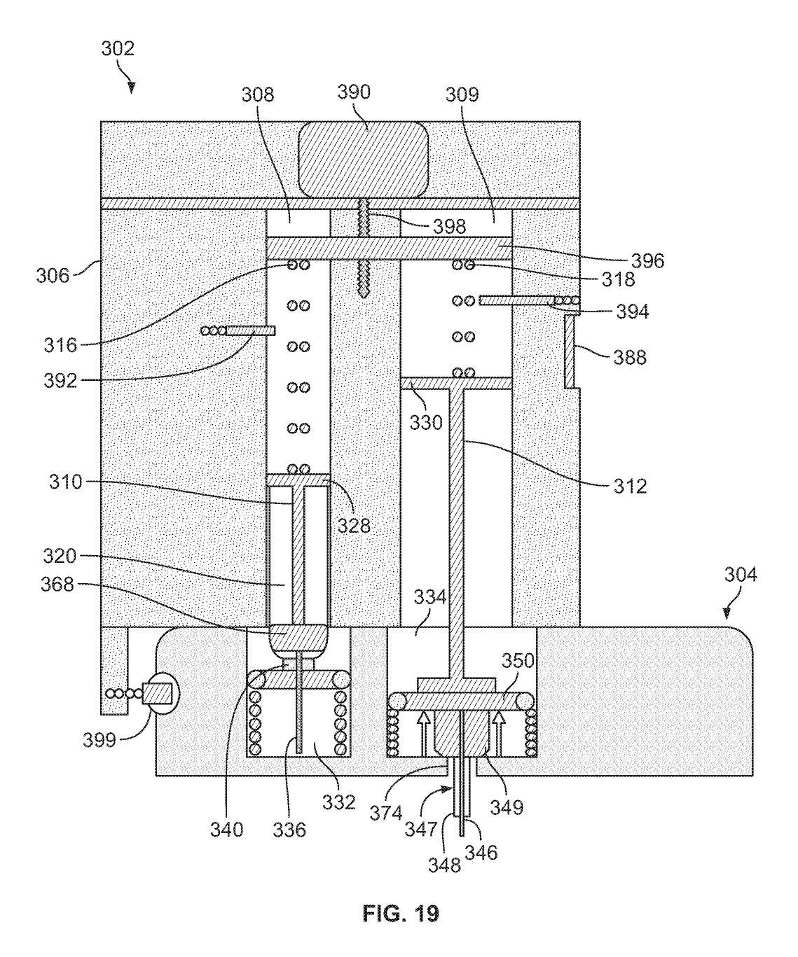

In various other embodiments, as shown, for example, in FIG. 19, either the fill driver 310, insert driver 312 (or, in some cases, both) are released upon mounting of the fill/insert device 302 to the delivery device 304. For example, connection of the spring loaded latch 399 with the delivery device 304 may generate a magnetic, electric, or mechanical response that results in release of the drivers 110, 112 (e.g., by retraction of retraction arms 392, 394).

In various other implementations of the third example embodiment, the fill/insert device 302 can be configured such that the fill driver 310 is released before the insert driver 312. As one example, the retraction button 388 can be configured to retract retractable arm 392 resulting in the release of the fill driver 310 first, which may cause the subsequent release of the insert driver 312. In still other implementations, both the fill driver 310 and the insert driver 312 can be released by the pressing of a retraction button (in some cases, separate retraction buttons; in other cases, the same retraction button) that causes a retraction arm attached to each driver to retract.

In various embodiments, once both the fill driver 310 and the insert driver 312 have reached the bottom of stroke and both the filling and inserting steps are completed, the fill/insert device 302 can be removed from the delivery device 304. The needle may be automatically retracted out of the patient's skin into the cannula deployment port 334 before the fill/insert device 302 is removed. The fill/insert device 302 can be removed from the delivery device 304 using any of the techniques previously described. In some cases, the latch 399 can be unloaded before the fill/insert device 302 is removed.

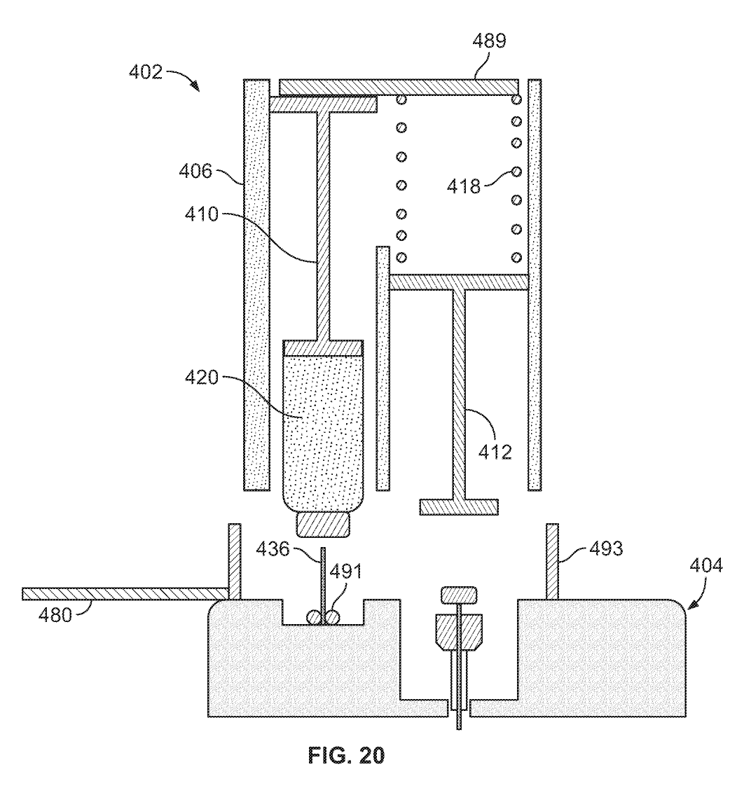

A fourth example embodiment is depicted in FIGS. 20-24. As shown, for example, in FIG. 20, a fill/insert device 402 can be provided to a patient pre-loaded with a medicament container, such as medicament vial 420. In other cases, a user can manually install the vial 420 into the fill/insert device 402, as described above. The fill/insert device 402 can feature a drive bar 489 coupled to a fill driver 410 and an insert driver 412. As depicted, in some instances, the drive bar 489 can be connected directly to the fill driver 410 and connected via a drive spring 418 to the insert driver 412.

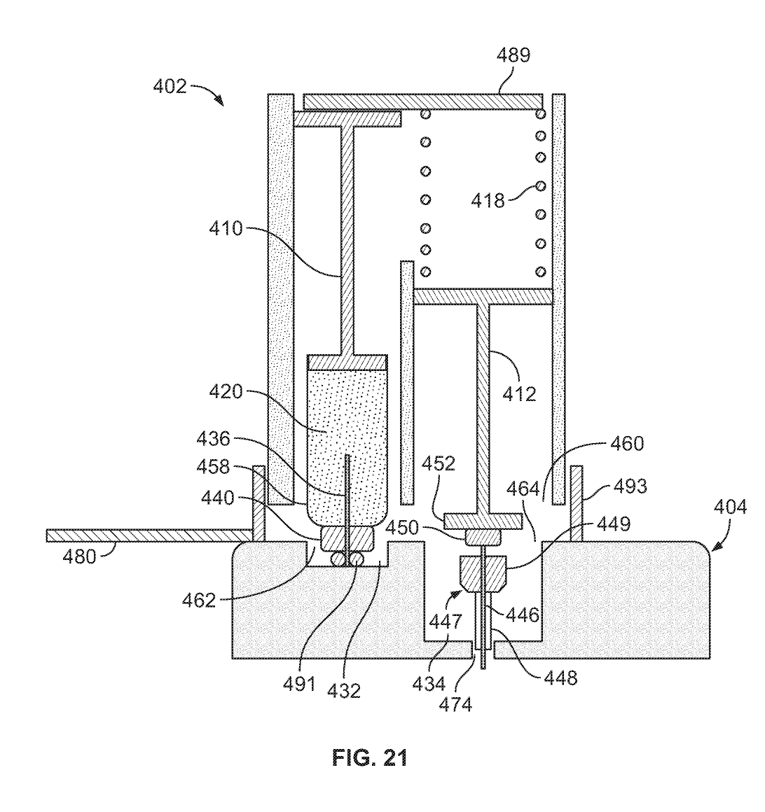

FIG. 21 is an example depiction of the fill/insert device 402 mounted to a medicament delivery device 404. The fill/insert device 402 can attach to the delivery device 404 using any techniques described previously. As another example, depicted for example in FIGS. 20-21, a housing 406 of the fill/insert device 402 can fit into or attach to a rim 493 detachably coupled to and extending upwards from the delivery device 404 (e.g., the rim 493 can fit into a groove on a top surface of the delivery device 404). Upon attachment (or in some cases, as the act of attachment), a hollow fill needle 436 in a fill port 432 can be guided into the interior of the medicament vial 420, so as to fluidically connect the medicament vial 420 with an interior of the delivery device 404. A fill bumper 491 can prevent a dispensing portion 440 of the medicament vial 420 from contacting a hard surface of the delivery device 404 (e.g., the bottom of the fill port 432), which could potentially damage the vial 420. In some embodiments, after the hollow fill needle 436 is guided into the vial 420 it becomes attached to the vial 420, e.g., such that the hollow fill needle 436 remains attached to the medicament vial 420 upon removal of the fill/insert device 402 from the delivery device 404. In general, the connection can be accomplished using any known technique/principle; for example, an interference fit between the hollow fill needle 436 and the dispensing portion 440.

Upon attachment, a drive portion 452 of the insert driver 412 may abut an insert interface 450 of a cannula delivery port 434. As in previous embodiments, the insert interface may be attached to a needle 446 that mates with a cannula assembly 447 that includes a base 449 and a cannula 448. In some instances, the drive portion 452 and insert interface 450 may become connected upon mounting. In general, the connection can be accomplished using any know technique (e.g., a magnetic force, compliant arms, loop and hook fasteners, etc.) As depicted, the drive spring 418 may be in an unloaded configuration when the fill/insert device 402 is initially mounted to the delivery device 404.

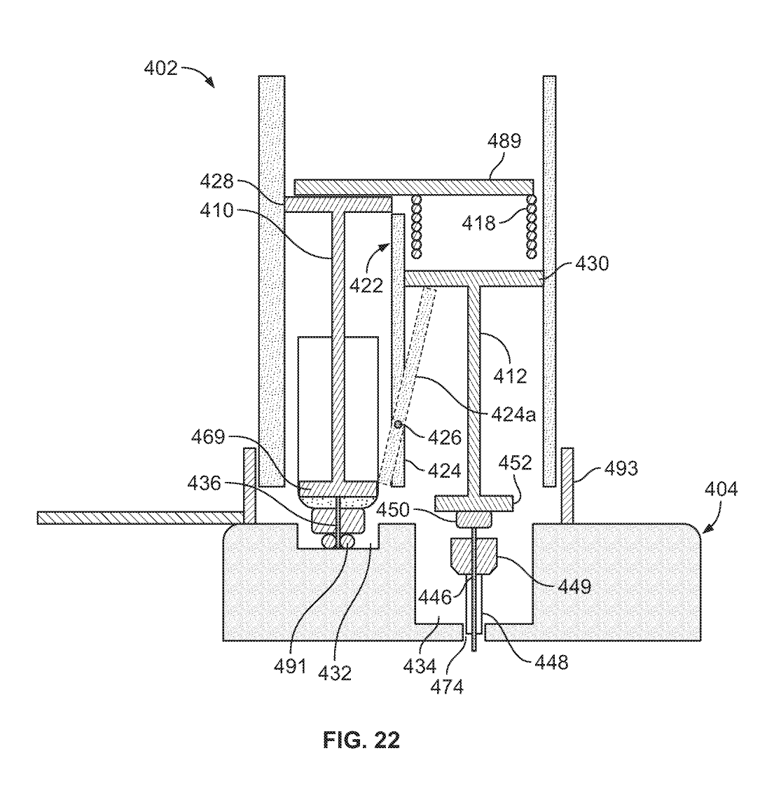

In various embodiments, after the fill/insert device 402 is mounted onto the delivery device 404, the drive bar 489 can be compressed downwards towards the delivery device 404 (see FIG. 22). The compressive force can be supplied manually by the user or from any other source. Compression of the drive bar 489 may perform the dual actions of (i) forcing a piston portion 469 of the fill driver 410 through a cylinder of the medicament vial 420 to force the medicament through the hollow fill needle 436 into a internal reservoir of the delivery device 404 (thereby filling the delivery device 404) and (ii) compressing the drive spring 418 into a loaded position. In general, the drive spring 418 can be held in the loaded position using any conventional technique, including the techniques described above with respect to other embodiments. For example, drive spring 418 can engage a catch 422, or a pivot bar 424 may be rotated or biased to hold an upper portion 430 of the insert driver 412. In FIG. 22, the pivot bar 424a in its rotated position is shown in phantom.

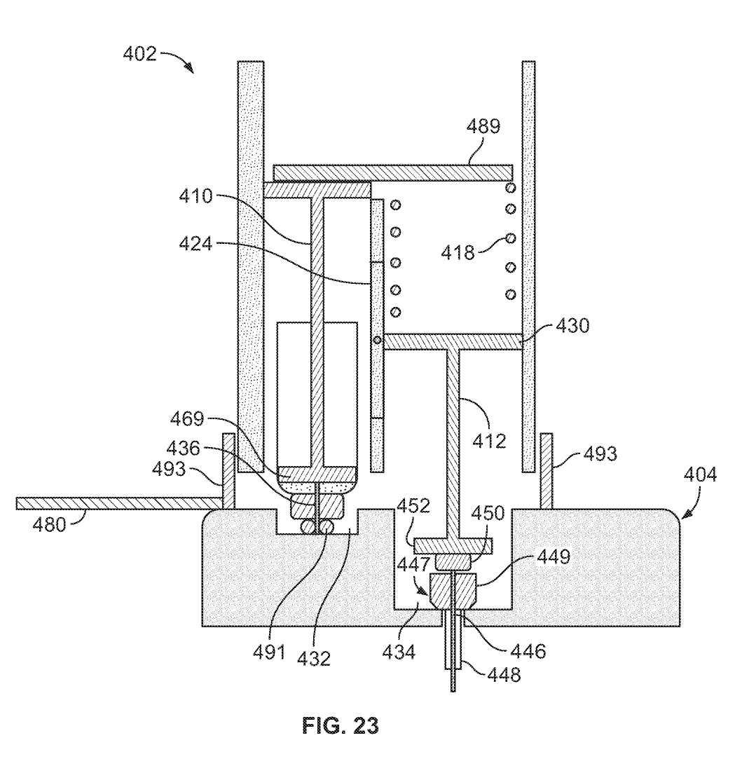

In various embodiments, translation of the fill driver 410 towards the delivery device 404 triggers the release of the insert driver 412 (see FIG. 23). For example, the upper portion 438 or piston portion 469 of the fill driver 410 can engage a linkage, or release a catch, holding the drive spring 418 in a loaded position. As another example, the upper portion 438 or piston portion 469 can rotate the pivot bar 424 to remove the resistance force it applies to the insert driver 412. In general, the insert driver 412 can be released at any point during the stroke of the fill driver 410; for example, at the beginning of the fill driver's stroke, or in some cases, at the end of the fill driver's stroke. Regardless of how the drive spring 418 is released, once it is, the drive spring 418 is free to expand and drive the insert driver 412, insert interface 450, needle 446, and cannula assembly 447 downwards, resulting in placement of the cannula 448, into the subcutaneous tissue of the patient. As described with respect to previous embodiments, this can result in the base being seated in the bottom of the cannula delivery port 434.

In various other implementations of this example embodiment, the fill/insert device 402 can be configured such that the insert driver 412 is released before the fill driver 410. As one example, as mentioned above, the insert driver 412 can be directly connected to the drive bar 489 and the fill driver 410 can be connected to the drive bar via a drive spring. In such instances, compression of the drive bar 489 can result in performance of the cannula inserting step and loading of the drive spring. Upon release of the drive spring, the filling step can occur.

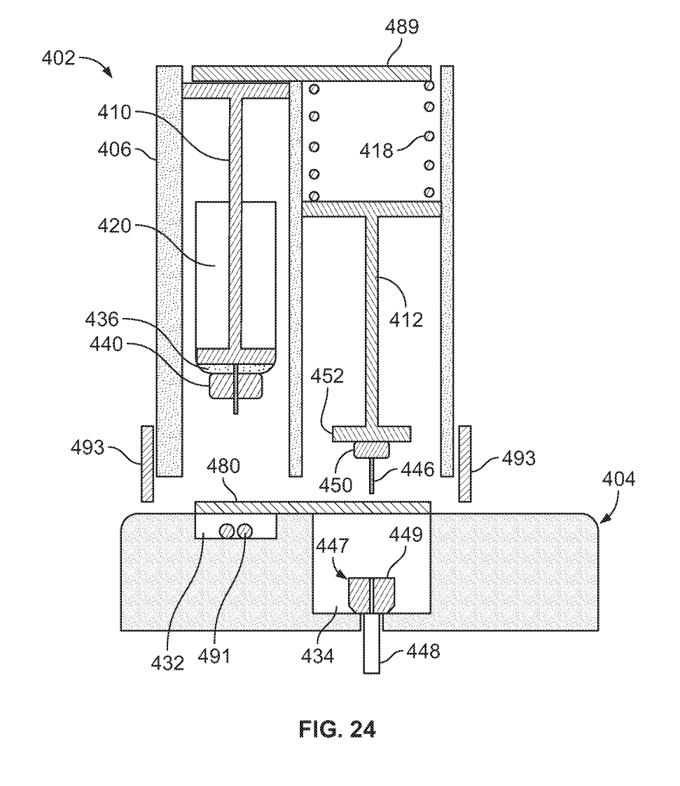

In various embodiments, once the insert driver 412 has reached the bottom of stroke and both the filling and inserting steps are completed, the fill/insert device 402 can be removed from the delivery device 404 (see FIG. 24). In some embodiments, the needle 446 can be automatically retracted out of the patient's skin into the cannula deployment port 434 before the fill/insert device 402 is removed. This can be accomplished using any of the techniques described above. The fill/insert device 402 can be removed from the delivery device 404 using any of the techniques previously described. For example, the patient may fully mate the indicator unit to the delivery device 404, resulting in a lid 480 translating over and closing off the top open surfaces of the fill port 432 and cannula delivery port 434. The closing of the lid 480 can force the rim 493 off of the medicament delivery device 404 (e.g., out of a groove) along with the fill/insert device 402. In some cases, the closing of the lid 480 can also provide a force that returns the drive bar 489 upwards (or in some cases translates it upwards a suitable distance) to remove any portions of the fill/insert device 402 from the fill port 432 (e.g., the medicament vial 420, dispensing portion 440, etc.) and the cannula delivery port 434 (e.g., the drive portion 452, etc.). Regardless of whether the drive bar 489 is forced upwards, the fill/insert device 402 can be manually removed from the delivery device 404 by the patient.

In instances in which the insert interface 450 has become connected to drive portion 452, the drive portion 452 and attached needle 446 can be removed from the delivery device 404 along with the fill/insert device 402, as shown in FIG. 24. Similarly, in instances in which the hollow fill needle 436 has become connected to the medicament vial 420, the hollow fill needle 436 can be removed from the delivery device 404 along with the fill/insert device 402. In various embodiments, one of, both of, or neither the hollow fill needle 436 and the needle 446 are removed along with the fill/insert device 402.

In various other implementations of any of the example embodiments described above (or other embodiments), both the filling step and inserting steps can be performed by a manual action of a user. As one example, a fill/insert device can include both a fill driver and an insert driver adapted to be driven by a manual force applied by the user. In some cases, both the fill driver and the insert driver are driven at the same time under the same force. In other cases, the fill driver and the insert driver are driven separately under separately-applied forces or sequentially under sequentially-applied forces.

The various example embodiments described above feature a hollow fill needle and a cannula insertion needle. The needles can be provided to a patient/user in a wide variety of ways. For example, one or both needles can be provided to the patient within the delivery device and remain in the delivery device after the filling and inserting steps. In some instances, one or both needles are provided to the patient within the delivery device and are removed into and with the fill/insert device when the fill/insert device is removed from the delivery device, after the filling and inserting steps are completed. In some instances, one or both needles are provided to the patient within the fill/insert device and remain within the delivery device when the fill/insert device is removed from the delivery device, after the filling and inserting steps are completed. In some instances, one or both needles are provided to the patient within the fill/insert device and remain within the fill/insert device when the fill/insert device is removed from the delivery device, after the filling and inserting steps are completed. In still other instances, one or both needles can be delivered to the patient as standalone components to be installed into the fill/insert device and/or the delivery device. In various embodiments, the hollow fill needle and cannula insertion needle can be provided and treated differently from one another.

Example additional benefits of using a combined fill/insert device used in the example embodiments described above can include some of the following. The fill/insert device can provide an ergonomic handle that can be useful, e.g., when removing an adhesive tape release liner from the base of the delivery device and before attaching the delivery device to a patient's skin, or to assist the user in compressing the delivery device against the skin. In some cases, the fill/insert device can include a force sensor that can determine whether a sufficient force has been applied for a sufficient time for the delivery device to be properly secured to the patient's skin. In some such cases, the patient can receive a visual, audible, and/or tactile feedback once this occurs; or, in some cases, if it does not occur. In some instances, the filling step can result in an automatic priming of the device. The fill/insert device may be able to collect an initial basal rate reading from the delivery device and communicate it to the indicator unit. In certain instances, the fill/insert device can be configured to provide feedback to the patient upon successful insertion of the cannula. For example, a sensor may be configured to determine if the cannula base has been seated properly in the bottom of the cannula delivery port and, if so, can cause an audible, visual, and/or tactile feedback to be provided to the user (similarly, a different feedback can be provided if cannula placement is unsuccessful). In certain embodiments, the fill/insert device can allow the delivery device to be filled from two separate cartridges. For example, a user may fill the device with a first cartridge using a conventional approach (e.g., piercing a septum with a needle), which may result in the internal reservoir only being partially filled. The user can then fill the delivery device using a second cartridge with the fill/insert device, as described above. In certain embodiments, the drive interface (and associated seal) may plug a filling pathway (e.g., where the microfluidic channels of the delivery device intersect with the cannula for delivery into the patient) better than conventional techniques. The fill/insert device may be reusable such that, after removal from a particular delivery device following completion of the filling and inserting steps, it can be reused with another (or, in some cases, the same) delivery device. In other instances, the fill/insert device is disposable and discarded after a single use.

The terms and expressions employed herein are used as terms and expressions of description and not of limitation and there is no intention, in the use of such terms and expressions, of excluding any equivalents of the features shown and described or portions thereof. In addition, having described certain embodiments of the invention, it will be apparent to those of ordinary skill in the art that other embodiments incorporating the concepts disclosed herein may be used without departing from the spirit and scope of the invention. The structural features and functions of the various embodiments may be arranged in various combinations and permutations, and all are considered to be within the scope of the disclosed invention. Unless otherwise necessitated, recited steps in the various methods may be performed in any order and certain steps may be performed substantially simultaneously. Accordingly, the described embodiments are to be considered in all respects as only illustrative and not restrictive. Furthermore, the configurations described herein are intended as illustrative and in no way limiting. Similarly, although physical explanations have been provided for explanatory purposes, there is no intent to be bound by any particular theory or mechanism, or to limit the claims in accordance therewith.

What is claimed is:

1. An apparatus for filling a medicament delivery device with medicament and inserting a cannula subcutaneously, the apparatus comprising:
a housing forming:
a first chamber portion comprising a first deployment mechanism; and
a second chamber portion comprising a second deployment mechanism;
a trigger mounted to the housing to activate at least one of the first deployment mechanism and the second deployment mechanism; and
an interface adapted to mate releasably with the medicament delivery device.

2. The apparatus of claim 1, wherein the medicament delivery device comprises an insulin pump attachable to skin of a user.

3. The apparatus of claim 1, wherein the first chamber is adapted to receive a medicament container.

4. The apparatus of claim 1, wherein the first deployment mechanism comprises a first driving member.

5. The apparatus of claim 1, wherein the first deployment mechanism comprises a piston portion.

6. The apparatus of claim 1, wherein the second deployment mechanism comprises a second driving member.

7. The apparatus of claim 1, wherein the second deployment mechanism comprises a drive portion.

8. The apparatus of claim 1, wherein the trigger is configured to activate both the first deployment mechanism and the second deployment mechanism simultaneously.

9. The apparatus of claim 1, wherein the trigger is configured to activate the first deployment mechanism and the second deployment mechanism sequentially.

10. The apparatus of claim 1, wherein the trigger comprises a manually actuated button.

11. The apparatus of claim 1, wherein the interface defines a first outlet corresponding with the first chamber portion and a second outlet corresponding with the second chamber portion.

12. A medicament filling and cannula insertion system, the system comprising:
a medicament delivery device; and
a medicament filling and cannula insertion apparatus comprising:
a first deployment mechanism for deploying a medicament container to fill the medicament delivery device with medicament; and
a second deployment mechanism for deploying a cannula subcutaneously to a user through the medicament delivery device.

13. The system of claim 12, wherein the medicament filing and cannula insertion apparatus further comprises:
a housing forming:
a first chamber portion comprising the first deployment mechanism; and
a second chamber portion comprising the second deployment mechanism; and
an interface adapted to mate releasably with the medicament delivery device, the interface forming a first outlet corresponding with the first chamber portion and a second outlet corresponding with the second chamber portion,
wherein the first outlet and the second outlet are arranged to correspond respectively with first and second inlets of the medicament delivery device when the apparatus is releasably attached thereto.

14. The system of claim 13, wherein the first inlet comprises a medicament fill port.

15. The system of claim 14, wherein the fill port comprises a spring and a fill needle.

16. The system of claim 15, wherein the spring biases an interface adapted to prevent inadvertent contact with the needle.

17. The system of claim 13, wherein the second inlet comprises a cannula delivery port.

18. The system of claim 17, wherein the cannula delivery port comprises a cannula, a needle, and a spring.

19. The system of claim 18, wherein the spring is biased to withdraw the needle after subcutaneous insertion of the cannula.

20. A method for filling a medicament delivery device and subcutaneously inserting a cannula into a patient, the method comprising the step of:
manually activating a trigger resulting in:
automatically filling the medicament delivery device with medicament; and
automatically inserting subcutaneously a cannula into the patient through the medicament delivery device.

21. The method of claim 20, wherein the step of manually activating the trigger comprises pushing a button.

22. The method of claim 20, wherein the step of filling the medicament delivery device comprises guiding a fill needle of the medicament delivery device into a medicament container.

23. The method of claim 22, wherein the step of filling the medicament delivery device further comprises forcing medicament from the medicament container into the medicament delivery device through the fill needle.

24. The method of claim 20, wherein the step of inserting subcutaneously a cannula comprises advancing the cannula and a needle through skin of the patient.

25. The method of claim 24, wherein the step of inserting subcutaneously a cannula further comprises retracting the needle from the cannula.

26. The method of claim 20, wherein the steps of filling the medicament delivery device and inserting subcutaneously the cannula occur sequentially.

27. The method of claim 26, wherein the step of filling the medicament delivery device is completed prior to inserting subcutaneously the cannula.

28. The method of claim 20, wherein the steps of filling the medicament delivery device and inserting subcutaneously the cannula occur simultaneously.
